(19) United States
(12) Patent Application Publication
(10) Pub. No.: US 2023/0176066 A1
(43) Pub. Date: Jun. 8, 2023

(54) PLASMON-ENHANCED FLUORO-DOT ASSAYS

(71) Applicant: Washington University, St. Louis, MO (US)

(72) Inventors: Anushree Seth, St. Louis, MO (US); Jingyi Luan, St. Louis, MO (US); Srikanth Singamaneni, St. Louis, MO (US)

(21) Appl. No.: 18/061,672

(22) Filed: Dec. 5, 2022

Related U.S. Application Data
(60) Provisional application No. 63/265,063, filed on Dec. 7, 2021.

(51) Int. Cl. G01N 33/58; G01N 33/543; G01N 21/64

(52) U.S. Cl. CPC G01N 33/582; G01N 33/543; G01N 21/648; G01N 21/6428; G01N 2333/525; G01N 2333/5412; G01N 2021/6439

(57) ABSTRACT

Described herein are methods and kits for performing plasmon-enhanced fluoro-dot assays. These assays enable observing a correlation between a chemical stimulus and a biological response of cultured cells in vitro.

### CROSS-REFERENCE TO RELATED APPLICATIONS

This application claims priority benefit of U.S. Provisional Application Ser. No. 63/265,063 filed Dec. 7, 2021, the entire contents of which are incorporated by reference herein.

### GOVERNMENT SUPPORT CLAUSE

This invention was made with government support under grant number CBET-1900277 awarded by the National Science Foundation. The government has certain rights in the invention.

### FIELD OF THE DISCLOSURE

The present disclosure is directed to methods and kits for performing plasmon-enhanced fluoro-dot assays. These assays enable observing a correlation between a chemical stimulus and a biological response of cultured cells in vitro.

### BACKGROUND OF THE DISCLOSURE

Proteins secreted from cells act as mediators of cell-cell communication, signaling and proliferation. Extracellular proteins facilitate cell migration and differentiation in cells. Proteins secreted by cells into the extracellular space constitute 13-15% of the entire proteome and include growth factors, cytokines, chemokines, antibodies, extracellular matrix proteins, enzymes, hormones, and antimicrobial peptides. Secretory proteins facilitate essential physiological and pathological processes such as cell-to-cell communication, cell signaling, activation, inflammation, coagulation, hemostasis, differentiation, migration, toxicity, and defense. Understanding the cell secretome is essential in numerous life sciences disciplines, including immunology, oncology, neurobiology, microbiology, endocrinology, and stem cell biology.

Currently, there is limited understanding of the identity, amount and function of secreted proteins at a single cell level. The emerging field of single-cell proteomics aims to overcome the need to deduce protein expression level from cellular mRNA levels. Presently, enzyme-linked immune-assay (ELISA) and mass spectrometry (MS)-based techniques serve as gold standard techniques to detect the presence and quantify the levels of protein in cell culture media or extra-cellular matrix. However, these methods fail to resolve the spatial and temporal secretion patterns of individual cells as they provide only a measure of the average response of a cell population. It is important to map the kinetics of these systematically coordinated secretion events at an ultra-high spatial resolution.

Researchers heavily rely on samples collected from cell culture supernatants and conventional immunoassays such as ELISA to assess the changes in proteins secreted by cells after various physicochemical or biological stimuli. Naturally, the concentrations of the target proteins in cell culture supernatants represent an ensemble average of the entire cell population (in vitro), thus masking the cell-to-cell heterogeneity and, more importantly, the locoregional correlation between biophysical stimulation and biochemical response. Further, for attaining a detectable signal in ELISA, one needs to collect supernatant from thousands of cells, which have been incubated for an extended duration, typically ranging from 12 hours to several days. Implicit averaging in these methods results in loss of information related to cell-to-cell heterogeneity, cell-to-cell interaction, and the spatial distribution of secreted proteins. This particularly confounds analysis when working with cells having sub-populations and multi-modal populations. Furthermore, due to the low sensitivity of these techniques, there is little information on the kinetics of protein secretion, particularly at early time-points after stimulation and under low levels of stimulation. It is imperative to detect and measure proteins secreted at single-cell resolution without having to rely on mRNA and mass spectrometry. In more than 60% of the cases, mRNA levels do not correlate with protein abundance.

Moreover, mass spectrometry requires specialized strategies to isolate and handle single cells and expensive instrumentation and training, making it unsuitable for routine single-cell secretome analysis. Although ELISpot and FluoroSpot have emerged as powerful tools for studying protein secretions at the single-cell level, they are semi-quantitative. The assay read-out is a colored or fluorescent “spot” on a white or dark background, either counted manually or with an ELISpot reader. Each spot, which is diffused at times, indicates a protein-secreting cell, and the size of the spot provides a qualitative and often vague estimate of the amount of protein secreted by the cell. In fact, several discrepancies in terms of variability and accuracy of these assays and how the data is interpreted obligated extensive efforts to establish standardized, automated guidelines for evaluation of ELISpot assays. Further, the inherently low signal warrants the use of specialized membrane-coated plates to retain more protein and long incubation time for a readable signal; the requirement for a dedicated reader further impedes its widespread applicability in laboratory settings.

The present disclosure aims to introduce a novel bioanalytical method that reveals the protein secretion patterns at a single cell level to understand the biophysical and biochemical correlation. The method improves on existing methods like ELISpot, such that instead of relying on a spot for indicating where a cell has secreted protein, individual fluorescent “dots” are revealed to indicate individual proteins within a “spot”.

The present disclosure is directed to methods and kits for performing plasmon-enhanced fluoro-dot assays. These assays enable observing a correlation between a chemical stimulus and a biological response of cultured cells in vitro.

### BRIEF DESCRIPTION OF THE DISCLOSURE

In one embodiment, the present disclosure is directed to a method for detecting cellular protein secretion, the method comprising: (i) adding at least one stimulant to a sample comprising at least one cell fixed on a substrate, wherein the at least one stimulant stimulates the at least one cell to secrete at least one protein; (ii) capturing the at least one protein secreted by the at least one cell; (iii) adding at least one detection antibody to the sample; (iv) adding at least one plasmonic-fluor to the sample; and (v) measuring at least one fluorescence signal from the sample.

In another embodiment, the present disclosure is directed to a kit comprising: a sample comprising a substrate, wherein the substrate optionally comprises at least one cell affixed thereon; at least one stimulant; at least one detection antibody; at least one plasmonic-fluor; and written instructions for detecting cellular protein secretion, comprising the steps of: (i) optionally affixing at least one cell on the substrate; (ii) adding the at least one stimulant to the sample, wherein the sample comprises at least one cell fixed on the substrate, wherein the at least one stimulant stimulates the at least one cell to secrete at least one protein; (iii) capturing the at least one protein secreted by the at least one cell; (iv) adding the at least one detection antibody to the sample; (v) adding the at least one plasmonic-fluor to the sample; and (vi) measuring at least one fluorescence signal from the sample.

### BRIEF DESCRIPTION OF THE DRAWINGS

FIG. 1A is an illustration of a step-by-step workflow for conventional ELISpot or FluoroSpot assays.

FIG. 1B is an exemplary embodiment of an illustration of a step-by-step workflow for a plasmon-enhanced Fluoro-dot assay relying on plasmonic-fluors as ultrabright biolabels in accordance with the present disclosure.

FIG. 1C depicts Cy5/DAPI merged epifluorescence microscopy images of a conventional fluor (Cy5) after JAWS II DCs were treated with 0, 20, 200 and 2000 ng/ml lipopolysachharide (LPS). Each panel contains 3 representative 20× images stitched together. The scale bar is 100 μm.

FIG. 1D depicts Cy5/DAPI merged epifluorescence microscopy images of a conventional quantum dot 655 after JAWS II DCs were treated with 0, 20, 200 and 2000 ng/ml lipopolysachharide (LPS). Each panel contains 3 representative 20× images stitched together. The scale bar is 100 μm.

FIG. 1E depicts Cy5/DAPI merged epifluorescence microscopy images of a Cy5-plasmonic-fluor in accordance with the present disclosure after JAWS II DCs were treated with 0, 20, 200 and 2000 ng/ml lipopolysachharide (LPS). Each panel contains 3 representative 20× images stitched together. The scale bar is 100 μm.

FIG. 1F depicts bright field images (left panel) and Cy5/DAPI merged images (right panel) of a single cell secreting TNFα, visualized using conventional fluor (Cy5), quantum dot 655, and Cy5-plasmonic-fluor in accordance with the present disclosure.

FIG. 1G depicts representative line-scans (left) and signal-to-noise ratio (SNR) (right) corresponding to conventional fluor (Cy5), quantum dot 655, and Cy5-plasmonic-fluor in accordance with the present disclosure.

FIG. 2A depicts Cy5 epifluorescence microscopy images of a fluoro-dot assay performed on JAWS II dendritic cells treated with a) 0, 2, 20, 200 and 2000 ng/ml lipopolysachharide (LPS) for 90 minutes using Cy5-plasmonic-fluor in accordance with the present disclosure, with a 100 μm scale bar (left) and Cy5/DAPI merged images of TNFα secreting cell visualized using Cy5-plasmonic-fluor at 60×, with a 50 μm scale bar (right).

FIG. 2B is a graphical illustration depicting the LPS treatment of JAWS II dendritic cells for studying the secretion levels of TNFα in a dose and time-dependent manner in accordance with the present disclosure.

FIG. 2C depicts Cy5 epifluorescence microscopy images of a fluoro-dot assay performed on JAWS II dendritic cells treated with a) 200 ng/ml of LPS treated for 20 minutes, 40 minutes, 60 minutes and 90 minutes using Cy5-plasmonic-fluor in accordance with the present disclosure, with a 100 μm scale bar (left) and Cy5/DAPI merged images of TNFα secreting cell visualized using Cy5-plasmonic-fluor at 60×, with a 50 μm scale bar (right).

FIG. 2D depicts quantification of the number of TNFα secreting cells with increasing dose of LPS in accordance with the present disclosure. Number of clusters were calculated using ten, 20× images for each treatment condition. ns: not significant, **p<0.01, ***p<0.001, ****p<0.0001 by one-way ANOVA and Tukey's post test.

FIG. 2E depicts quantification of the amount of TNFα secreted by individual cells with increasing dose of LPS in accordance with the present disclosure. Number of particles/cluster were calculated using ten, 20× images for each treatment condition. ns: not significant, **p<0.01, ***p<0.001, ****p<0.0001 by one-way ANOVA and Tukey's post test.

FIG. 2F depicts quantification of the number of TNF. secreting cells with increasing duration of LI'S treatment in accordance with the present disclosure. Number of clusters were calculated using ten, 20× images for each treatment condition. ns: not significant, **p<0.01, ***p<0.001, ****p<0.0001 by one-way ANOVA and Tukey's post test.

FIG. 2G depicts quantification of the amount of TNfα secreted by individual cells with increasing duration of LPS treatment in accordance with the present disclosure. Number of particles/cluster were calculated using ten, 20× images for each treatment condition. ns: not significant, **p<0.01, ***p<0.001, ****p<0.0001 by one-way ANOVA and Tukey's post test.

FIG. 3A depicts a multiplexed analysis of secretion of TNFα and IL-6 from single JAWS II DC when subjected to 200 ng/ml LPS after 30 min, 1 hour, 2 hours, and 3 hours of incubation in accordance with the present disclosure. Epifluorescence microscopy images of the cells using TNFα detection antibody conjugated cy5-plasmonic-fluor (red) and IL-6 detection antibody conjugated cy3-plasmonic-fluor (yellow) overlaid with DAPI nuclei stain (blue). Each panel contains four representative 20× images stitched together. The scale bar is 100 μm.

FIG. 3B depicts a quantification of both TNFα and IL-6 from individual JAWS II DC after LPS treatment for 30 minutes, 1 hour, 2 hours, and 3 hours in accordance with the present disclosure. Number of clusters and number of particles/cluster were calculated using seven, 20× images for each treatment condition.

FIG. 3C depicts representative higher magnification images (60×) of JAWS II DCs showing the multiplexed detection of the TNFα and IL-6 secretion and the associated cell-to-cell heterogeneity indicated by white arrows in accordance with the present disclosure. The scale bar is 50 μm.

FIG. 4A depicts scanning electron microscopic images (left) and epifluorescence microscopy images (right) from the same regions of substrate drop-casted with plasmonic-fluors showing one-to-one correlation of individual plasmonic-fluors between the two images in accordance with the present disclosure. The scale bar is 5 μm.

FIG. 4B depicts transmission electron microscopy images of streptavidin Cy5-plasmonic-fluor and streptavidin Cy3-plasmonic-fluor in accordance with the present disclosure.

FIG. 4C depicts fluorescence intensity of Cy5/Cy3-conventional fluor and Cy5/Cy3-plasmonic-fluor at their different molar concentrations in accordance with the present disclosure. Data are represented as mean±s.d (n=2 repeated tests).

FIG. 5A depicts density-dose dependence images demonstrating increasing density of plasmonic-fluors with increasing concentration of cytokine TNFα, IL-6 and IFNγ in accordance with the present disclosure. The scale bar is 100 μm.

FIG. 5B depicts standard curves for the plasmon-enhanced fluorescence-linked immunosorbent assay (p-FLISA) for TNFα, IL-6 and IFNγ in accordance with the present disclosure. The data were fit using 4-parameter logistic (4 PL) sigmoidal curve. Limit of detection (LOD) is defined as (mean+3 σ) of the blank.

FIG. 6A depicts a fluoro-dot assay performed on JAWS II DCs treated with 0, 2, 20, 200, and 2000 ng/ml LPS for 90 minutes in accordance with the present disclosure. Cy5 epifluorescence microscopy images of the assay using Cy5-plasmonic-fluor. Each panel contains eight representative 20× images stitched together. The scale bar is 100 μm.

FIG. 6B depicts a fluoro-dot assay performed on JAWS II DCs treated with 200 ng/ml of LPS treated for 20 minutes, 40 minutes, 60 minutes, and 90 minutes in accordance with the present disclosure. Cy5 epifluorescence microscopy images of the assay using Cy5-plasmonic-fluor. Each panel contains eight representative 20× images stitched together. The scale bar is 100 μm.

FIG. 7A depicts TNFα secretion analyzed by ELISA of the supernatant of JAWS II DCs after stimulation of LPS for 90 minutes with 0, 2, 20, 200 and 2000 ng/ml in accordance with the present disclosure.

FIG. 7B depicts TNFα secretion analyzed by ELISA of the supernatant of JAWS II DCs after stimulation of LPS for 20, 40, 60, and 90 minutes with 200 ng/ml, n=2 in accordance with the present disclosure.

FIG. 8 depicts a representative example of the image processing algorithm used for determining the number of clusters and number particles in each cluster in accordance with the present disclosure.

FIG. 9A depicts representative Cy5 (red) and DAPI (blue) merged images revealing details of TNFα secretion in isotropic secretion in accordance with the present disclosure. The scale bar is 50 μm.

FIG. 9B depicts representative Cy5 (red) and DAPI (blue) merged images revealing details of TNFα secretion in anisotropic secretion in accordance with the present disclosure. The scale bar is 50 μm.

FIG. 9C depicts representative Cy5 (red) and DAPI (blue) merged images revealing details of TNFα secretion in secretion by two cells in proximity (doublet secretion) in accordance with the present disclosure. The scale bar is 50 μm.

FIG. 10A depicts a schematic illustration depicting the step-by-step method used for validation of specificity of antibody-conjugated plasmonic-fluor by spotting capture antibodies of both cytokines (TNFα and IL-6) in the same well of a microtiter plate in accordance with the present disclosure.

FIG. 10B depicts epifluorescence images of antibody-conjugated plasmonic fluors for their respective cytokine: TNFα (top-left) and IL-6 (top-right) and both TNFα and IL-6 (bottom-middle) in accordance with the present disclosure. The top images were collected using a 4× objective, and the grey-boxes shown in 4× images were taken using the 20× objective.

FIG. 11A depicts density-dose dependence images demonstrating increasing density of plasmonic-fluors with increasing concentration of cytokine TNFα, IL-6 in a multiplexed assay in accordance with the present disclosure. The scale bar is 100 μm.

FIG. 11B depicts standard curves for the multiplexed plasmon-enhanced fluorescence-linked immunosorbent assay (p-FLISA) for TNFα and IL-6 in accordance with the present disclosure. The data were fit using a 4-parameter logistic (4 PL) sigmoidal curve. Limit of detection (LOD) is defined as (mean+3 α) of the blank.

### DETAILED DESCRIPTION OF THE DISCLOSURE

Described herein is a new ultrasensitive method for detecting and quantifying protein secretion at a single-cell level. The high sensitivity of the assay stems from an ultrabright plasmon-enhanced fluorescent nanolabel called plasmonic-fluor. The enhancement of the emission of fluorophores in proximity to plasmonic nanostructures such as gold and silver nanoparticles is largely attributed to (i) the enhanced electromagnetic field at the surface of the plasmonic nanoparticles; and (ii) the decrease in the fluorescence lifetime due to the coupling between the excited fluorophores and the surface plasmons of the nanoparticles.

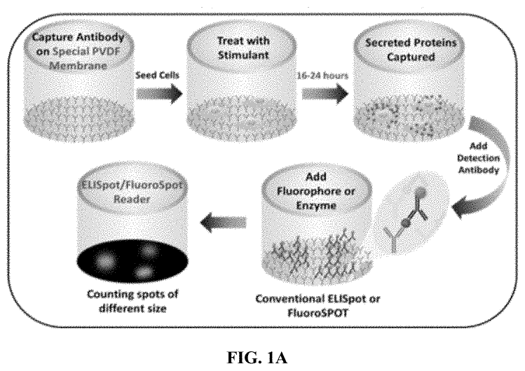

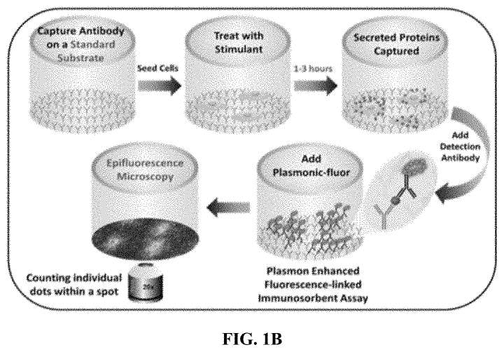

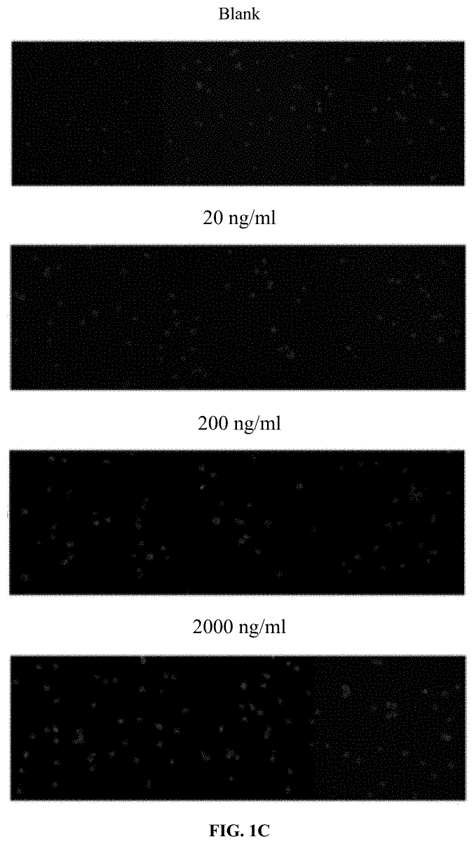

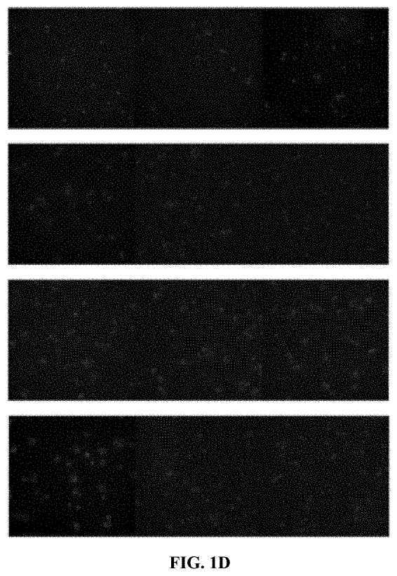

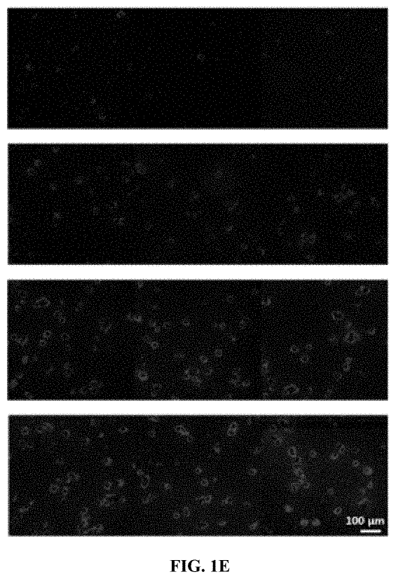

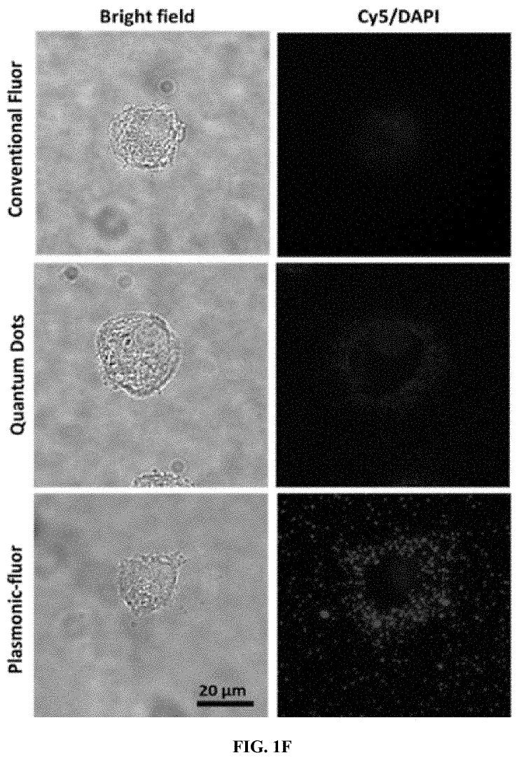

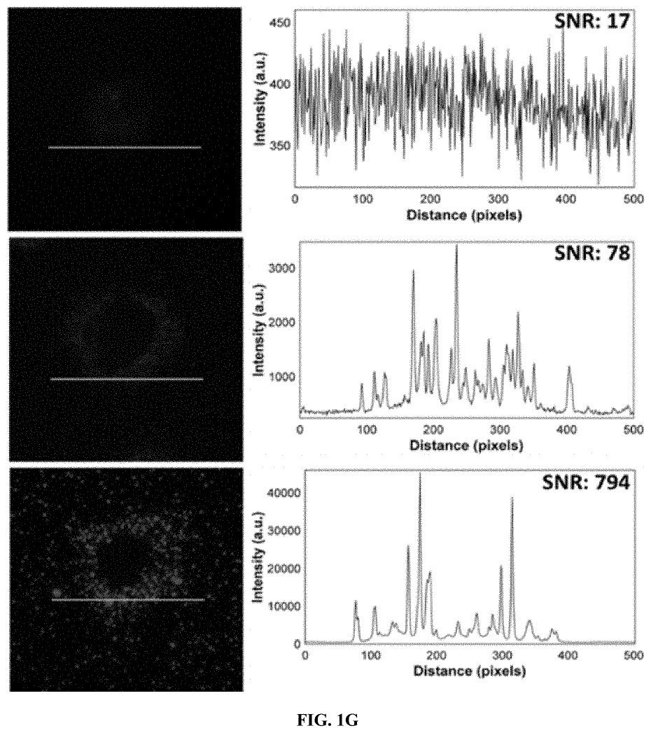

The plasmon-enhanced method reported herein, called “Fluoro-dot assay”, is better than conventional approaches as (i) it can be performed on readily available glass-bottom 96-well plates, unlike special PVDF/nitrocellulose membrane coated plates; (ii) it can detect protein secretions within 30 minutes to a few hours, as compared to overnight or days of incubation; (iii) the assay can be read using a standard epifluorescence microscope with a 20× objective; (iv) it provides spatial information at cellular and sub-cellular scales; and (v) the assay read-out is digital and quantitative. The read-out is in the form of images displaying distinguishable dots which can be quantified rather than the vaguely defined spot in ELISpot and FluoroSpot assays. Significantly, in this assay, the cells can be fixed on the plate so the spatial information such as the morphology of the cells, directionality or polarized secretion, activation of fluorescent reporters, and/or functional significance of heterogeneity can be correlated with protein secretion (FIG. 1B). For example, during innate immune responses, macrophages respond to a microbial challenge by inducing a variety of cytokines, including TNFα, IL-10, and IFN-α/(3, amongst others. However, it has been challenging to assess whether secretion is driven by the majority of directly infected cells, a select subset of infected cells, or uninfected cells in the population. Most previous attempts to unravel single-cell protein secretion utilized microfluidics-based assays (micro-/nano-wells, micro-troughs, droplet platforms) or hyperspectral imaging. These methods have not been widely adopted as mainstream tools due to the compartmentalization of cells, which hinders or eliminates the interaction between cells for natural and orchestrated protein secretion. Microfluidics-based assays also involve sophisticated tailoring of imaging platforms and optimizing the assay to suit the read-out format. Attempts to visualize single-cell secretion using methods such as interferometric detection of scattered light (iSCAT), gold nanohole array-based opto-fluidic devices and surface-enhanced Raman spectroscopy (SERS)-plasmonic colloidosome-based detectors require complex optical set-up and are limited by low-resolution, yielding imprecise images of protein secretion.

Using an ultrabright and specific fluorescent nanolabel described herein, low to high abundant proteins secreted by single cells can be imaged. The assay has been extensively validated by probing proteins secreted from dendritic cells (DCs) in response to TLR4 stimulation. This is the first disclosure demonstrating high resolution images of single-cell protein secretion, revealing previously inaccessible details such as cell-to-cell heterogeneity and directionality.

In many embodiments, methods according to the present disclosure are suitable for detecting cellular protein secretion.

In some embodiments, the method for detecting cellular protein secretion comprises: (i) adding at least one stimulant to a sample comprising at least one cell fixed on a substrate, wherein the at least one stimulant stimulates the at least one cell to secrete at least one protein; (ii) capturing the at least one protein secreted by the at least one cell; (iii) adding at least one detection antibody to the sample; (iv) adding at least one plasmonic-fluor to the sample; and (v) measuring at least one fluorescence signal from the sample.

In many embodiments, the at least one stimulant is selected from any suitable stimulant known in the art. In some embodiments, the at least one stimulant is selected from the group consisting of toll-like receptor 4 (TLR4) agonists, lipopolysaccharide (LPS), and combinations thereof.

In many embodiments, the at least one detection antibody is selected from any suitable detection antibody known in the art. The at least one detection antibody is natural, artificial, and/or modified. In some embodiments, the at least one detection antibody is biotinylated.

In many embodiments, the at least one plasmonic-fluor is selected from any suitable plasmonic-fluor known in the art. Suitable plasmonic-fluors are described in U.S. patent application Ser. No. 17/281,480, which is herein incorporated by reference in its entirety.

In some embodiments, the at least one plasmonic-fluor is streptavidin-conjugated. In some embodiments, the at least one plasmonic-fluor comprises a plasmonically active material selected from the group consisting of gold (Au), silver (Ag), copper (Cu), and combinations thereof.

In some embodiments, the at least one plasmonic-fluor comprises a plasmonic nanostructure selected from the group consisting of nanorods, nanocubes, nanospheres, bimetallic nanostructures, gold core silver shell nanocuboids, nanotubes, gold nanorods, silver nanocubes, silver nanospheres, gold nanorod core, silver shell (AuNR®Ag) nanocuboids, nanostructures with sharp tips, nanostars, hollow nanostructures, nanocages, nanorattles, nanobipyramids, nanoplates, self-assembled nanostructures, nanoraspberries, and combinations thereof.

In some embodiments, the at least one plasmonic-fluor comprises a fluorescent label selected from the group consisting of Cy3, Cy5, and combinations thereof. In some embodiments, the at least one plasmonic-fluor is selected from the group consisting of Streptavidin-conjugated Cy3-plasmonic-fluor (PF550™ ultrabright fluor), streptavidin-conjugated Cy5-plasmonic-fluor (PF650™ ultrabright fluor), and combinations thereof.

In many embodiments, the substrate is selected from any suitable substrate known in the art. In some embodiments, the substrate is selected from the group consisting of glass substrates, plastic substrates, well-plates, and combinations thereof.

In many embodiments, the fluorescence signal is measured according to any suitable technique known in the art. In some embodiments, the method step of measuring at least one fluorescence signal from the sample comprises measuring the at least one fluorescence signal with a technique selected from the group consisting of fluorescence microscopy, epifluorescence microscopy, and combinations thereof. In some embodiments, the method step of measuring at least one fluorescence signal from the sample comprises measuring a plurality of fluorescence signals from a plurality of fluorescent dots.

In many embodiments, the at least one cell is selected from any suitable cell and/or cell line known in the art. In some embodiments, the at least one cell is selected from the group consisting of dendritic cells, JAWS II cells, and combinations thereof.

In many embodiments, the at least one protein is selected from any suitable protein known in the art. In some embodiments, the at least one protein secreted by the at least one cell is selected from the group consisting of cytokines, TNFα, IFN-α/β, IL-6, IFNγ, and combinations thereof.

In some embodiments, the method further comprises measuring at least one spatial signal from the sample, wherein the at least one spatial signal is measured with a technique selected from the group consisting of microscopy, scanning electron microscopy (SEM), transmission electron microscopy (TEM), and combinations thereof.

In some embodiments, the method further comprises incubating the sample.

In many embodiments, methods according to the present disclosure are performed according to the supplies and instructions of a kit. In some embodiments, the kit comprises: a sample comprising a substrate, wherein the substrate optionally comprises at least one cell affixed thereon; at least one stimulant; at least one detection antibody; at least one plasmonic-fluor; and written instructions for detecting cellular protein secretion, comprising the steps of: (i) optionally affixing at least one cell on the substrate; (ii) adding the at least one stimulant to the sample, wherein the sample comprises at least one cell fixed on the substrate, wherein the at least one stimulant stimulates the at least one cell to secrete at least one protein; (iii) capturing the at least one protein secreted by the at least one cell; (iv) adding the at least one detection antibody to the sample; (v) adding the at least one plasmonic-fluor to the sample; and (vi) measuring at least one fluorescence signal from the sample.

In some embodiments, the sample comprises at least one cell fixed on the substrate, wherein the at least one cell is selected from the group consisting of at least one cell fixed to the substrate provided with the kit, at least one cell fixed to the substrate by a user, and combinations thereof.

### EXAMPLES

Without further elaboration, it is believed that one skilled in the art using the preceding description can utilize the present disclosure to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Example 1. Experimental Methods

Plasmonic-fluor procurement and characterization.

Streptavidin-conjugated Cy3-plasmonic-fluor (PF550™ ultrabright fluor) and streptavidin-conjugated Cy5-plasmonic-fluor (PF650™ ultrabright fluor) were purchased from Auragent Biosceince LLC (St. Louis, USA). The extinction was measured using a Shimadzu UV-1800 spectrophotometer. SEM images were obtained using a FEI Nova 2300 field-emission scanning electron microscope at an acceleration voltage of 10 kV. TEM images were obtained using a JEOL JEM-2100F field emission instrument. A drop of aqueous solution was dried on a carbon-coated grid, which had been made hydrophilic by glow discharge. Molar concentrations of plasmonic-fluors was calculated as described previously. Fluorescence intensity was recorded using Azure Biomolecular Imager: Sapphire RGBNIR (Azure Biosystems, Inc. Dublin, USA) and the images were analyzed using Licor Image Studio Lite.

Standard Curve Using Plasmonic-Fluors.

Mouse TNFα DuoSet ELISA kit (R&D systems, catalog number DY410, lot number P189768), mouse IL-6 DuoSet ELISA kit (R&D systems, catalog number DY406, lot number P234212), mouse IFNγ DuoSet ELISA kits (R&D systems, catalog number DY485-05, lot number P234214) and mouse IL-1β (Invitrogen, catalog number 88-7013-88 and lot number 183204000) were used to perform the assays. Glass-bottom 96-well black plate (P96-1.5H-N, Cellvis, Mountain View, USA) was first coated with capture antibodies as per manufacturer's instructions (100 μl/well) and incubated overnight at 4° C. The plate was washed three times with 1×PBST (1×PBS with 0.05% Tween-20) and then blocked with 200 μl of reagent diluent (1×PBS in 1% BSA, 0.2 μm filtered). After blocking the plates were washed three times with PBST, and serial dilutions of standard protein was added to different wells in duplicates and incubated for 2 hours at room temperature. The plates were washed three times with PBST and then incubated for 2 hours with 100 μl of biotinylated detection antibody as per the manufacturer's instructions. The plates were washed three times with PBST and streptavidin Cy5-plasmonic-fluor (extinction 0.5) for 30 minutes at room temperature in dark. Finally, the plates were washed three times in PBST and imaged using Nikon TsR2 epifluorescence microscope.

Fluoro-Dot Assay on JAWS II DC.

Mouse TNFα DuoSet ELISA kits (R&D systems, catalog number DY410, lot number P189768) was used to perform the assays. Glass-bottom 96-well black plate (P96-1.5H-N, Cellvis, Mountain View, USA) was first coated with capture antibodies (0.8 μg/ml in PBS), 100 μl/well and incubated overnight at 4° C. The plate was washed three times with 1×PBS and then blocked with 200 μl of reagent diluent (1×PBS in 1% BSA, 0.2 μm filtered). After blocking, the plates were washed three times with PBS. Mouse JAWS II dendritic cells (immature, monocytes ATCC® CRL11904™) were cultured in alpha minimum essential medium with ribonucleosides, deoxyribonucleosides, 4 mM L-glutamine, 1 mM sodium pyruvate and 5 ng/ml murine GM-CSF, 10% heat-inactivated fetal bovine serum, 50 IU/ml of penicillin, 50 μg/ml of streptomycin. JAWS II DCs were seeded on the capture coated plates, at the seeding density of 5,000 cells/well in 100 μl of medium followed by incubation at 37° C. in 5% CO₂ for 30 minutes. All the non-adherent cells were removed by taking out the medium and replacing it with 100 μl of fresh medium containing varying amounts of LPS ranging from (0 to 2000 ng/ml). The cells were incubated at 37° C. in 5% CO₂ for varying durations from 20 minutes to 90 minutes.

After completion of the incubation duration, the medium was decanted, and the cells were fixed using 100 μl/well of 4% neutral buffered formalin (NBF) for 20 minutes at room temperature. The plates were then washed three times with PBS and incubated with biotinylated detection antibodies, 75 ng/ml in reagent diluent for 2 hours at room temperature. The plates were washed three times using PBS and then were incubated with 100 μl/well of streptavidin Cy5-plasmonic-fluors (extinction 0.5) for 30 minutes at room temperature in dark. For comparison, 100 μl of 1 μg/ml of streptavidin-Cy5 (Thermo Fisher Scientific, catalog number SA1011) as conventional fluorophore and 1 nM of streptavidin QD655™ (Thermo Fisher Scientific, catalog number Q10123MP) was added to each well for 30 minutes at room temperature in dark. The plates were washed with PBS three times, and the nuclei of the cells were stained with 300 nM DAPI solution (Millipore Sigma, St. Louis, Mo., USA) for 5 minutes at room temperature in dark. Finally, the plates were washed three times in PBS and imaged using Nikon TsR2 epifluorescence microscope. For ELISA, culture supernatants of JAWS II DC after treatment described above were collected and TNFα concentration was measured using mouse TNFα DuoSet ELISA kits (R&D systems, catalog number DY410, lot number P189768) as per manufacturer's instructions.

Epifluorescence Microscopy.

All images were acquired on a Nikon Eclipse Ts2R-FL epifluorescence illumination microscope with a 20×, 0.75—numerical aperture (NA) lens and 60×, 1.4-NA. The microscope is attached to Hamamatsu digital camera (ORCA-Flash 4.0) with aura light engine. NIS-Elements AR 5.11.01 64-bit software was used to acquire images. Bright field and fluorescence images were collected in four channels corresponding to DAPI, Cy5, TRITC and GFP. For Cy5, TRITC and GFP, 200 ms exposure time was used and for DAPI exposure time of 50 ms was used. All images were saved as .tif files and further processed.

Image Processing and Calculation of Signal-to-Noise Ratio.

Image J 1.53a (64-bit), was used for adjusting the brightness and contrast of .tif images. Pseudo-color was imparted to images collected from different channels and merged using Image J tool. Pseudo-line was drawn on the image and analyzed using “plot profile” feature to obtain intensity vs. distance (pixels) graph. The raw data of the graph was exported, and signal-to-noise ratio (SNR) was calculated using the following equation:

SNR=(Average signal)/(Standard deviation of noise)

Average of 5 pixels with highest intensity was recorded as average signal. Standard deviation of first and last 50 pixels was recorded as standard deviation of noise.

Antibody Conjugation on Plasmonic-Fluor for Multiplexing and Validation.

Streptavidin-conjugated Cy3-plasmonic-fluor (40 extinction 32) and streptavidin-conjugated Cy5-plasmonic-fluor (40 extinction 30) was added to 50 μl of 4.5 μg/ml biotinylated IL-6 detection antibody and biotinylated TNF-α detection antibody, respectively. The mixture was incubated for 30 minutes at room temperature and then washed twice with pH 10 water. For washing, Cy3-plasmonic-fluor was centrifuged at 4,000 revolutions per minute (rpm) for 10 minutes, and Cy5-plasmonic-fluor was centrifuged at 6,000 rpm for 10 minutes. Finally, the pellet was resuspended in 1% BSA in 1×PBS and stored in 4° C. until further use. In order to validate the successful conjugation of the antibody, 0.5 μl of TNFα capture antibody (0.8 μg/ml in 10% glycerol in 1×PBS) was deposited on top-left area of a 96-well glass-bottom plate. Similarly, 0.5 μl of IL-6 capture antibody (2 μg/ml in 10% glycerol in 1×PBS) was deposited on top-right area of the same 96-well glass-bottom plate. 0.5 μl of a mixture of TNFα capture antibody (0.8 μg/ml in 10% glycerol in 1×PBS) and IL-6 capture antibody (2 μg/ml in 10% glycerol in 1×PBS) was deposited on bottom-middle area of the same 96-well glass-bottom plate. The plate was sealed with plate sealant and incubated for 2 hours at room temperature. The plate was washed three times with 1×PBST and then blocked with 200 μl of reagent diluent (1×PBS in 1% BSA, 0.2 μm filtered). After blocking, the plates were washed three times with PBST and 5,000 μg/ml of both of standard proteins (TNFα and IL-6) in 100 μl of 1% BSA in 1×PBS was added to the well and incubated for 2 hours at room temperature. The plates were washed three times with PBST and then incubated for 2 hours with a suspension comprised of 50 μl of IL-6 detection antibody-conjugated Cy3-plasmonic-fluor and 50 μl of TNFα detection antibody-conjugated Cy5-plasmonic-fluor (extinction 1 each) in dark. Finally, the plates were washed three times in PBST and imaged using Nikon TsR2 epifluorescence microscope using a 4× objective.

Multiplexed Fluoro-Dot Assay.

Mouse TNFα DuoSet ELISA kits (R&D systems, catalog number DY410, lot number P189768) and mouse IL-6 DuoSet ELISA kit (R&D systems, catalog number DY406, lot number P234212) were used to perform the assays. Glass-bottom 96-well black plate (P96-1.5H-N, Cellvis, Mountain View, USA) was first coated with both TNFα capture antibody (0.8 μg/ml in PBS) and IL-6 capture antibody (2 μg/ml in PBS), 100 μ/well, and incubated overnight at 4° C. The plate was washed three times with 1×PBS and then blocked with 200 μl of reagent diluent (1×PBS in 1% BSA, 0.2 μm filtered). After blocking the plates were washed three times with PBS. JAWS II dendritic cells (ATCC® CRL-11904™) were cultured in alpha minimum essential medium with ribonucleosides, deoxyribonucleosides, 4 mM L-glutamine, 1 mM sodium pyruvate and 5 ng/ml murine GM-CSF, 10% heat-inactivated fetal bovine serum, 50 IU/ml of penicillin, 50 μg/ml of streptomycin. JAWS II DCs were seeded on the capture coated plates, at the seeding density of 5,000 cells/well in 100 μl of medium followed by incubation at 37° C. in 5% CO₂ for 30 minutes. All the non-adherent cells were removed by taking out the medium and replacing it with 100 μl of fresh medium containing 200 ng/ml of LPS. The cells were incubated at 37° C. in 5% CO₂ for 30 minutes, 1 hour, 2 hours, and 3 hours. The wells with medium and without LPS were incubated for 3 hours. Subsequently, the medium was decanted, and cells were fixed using 100 μl/well of 4% neutral buffered formalin (NBF) for 20 minutes at room temperature. The plates were then washed three times with PBS and incubated with biotinylated detection antibody conjugated plasmonic-fluors (TNFα-detection antibody conjugated Cy5-plasmonic-fluor and IL-6 detection antibody conjugated Cy3-plasmonic-fluor: extinction 0.5) in reagent diluent for 2 hours at room temperature. The plates were washed three times using PBS and the nuclei of the cells were stained with 300 nM DAPI solution (Millipore Sigma, St. Louis, Mo., USA) for 5 minutes at room temperature in dark. Finally, the plates were washed three times in PBS and imaged using Nikon TsR2 epifluorescence microscope.

Statistics.

For analyzing the statistical difference between more than two groups, one-way analysis of variance (ANOVA) with a post-hoc Tukey's honest significance test was used. Statistical significance of the data was calculated at 95% (p<0.05) confidence intervals. All values are expressed as mean±standard deviation. GraphPad Prism 6 was used for all statistical analysis. Linear regression was used to calculate the equation and to derive the slope of fluorescence intensity vs. molar concentration graph. 4-parameter logistic (4-PL) was used to calculate the R² values and LOD in the standard curves of immuno-assays. The LOD is defined as the analyte concentration corresponding to the mean fluorescence intensity of blank plus three times of its standard deviation (mean+3σ). For Fluoro-dot assay, all measurements were taken from distinct samples as well as different regions of the same sample.

### Example 2. Fluoro-Dot Assay: Comparison with Conventional Fluorophores and Quantum Dots

First, plasmonic-fluor performance was compared with that of conventional fluorophores and quantum dots to enable the detection of proteins secreted by single cells. For this purpose, a dendritic cell line (JAWS II) was used, which secretes the pro-inflammatory cytokine TNFα after stimulation with toll-like receptor 4 (TLR4) agonist, lipopolysaccharide (LPS), in a dose-dependent manner. These cells were seeded on a TNFα capture antibody-coated glass-bottom 96-well plate followed by LPS treatment at three different concentrations (20 ng/ml, 200 ng/ml, and 2000 ng/ml). After 90 minutes of incubation, the cells were fixed and incubated with biotinylated TNFα detection antibody. Different wells were then treated with streptavidin-Cy5, streptavidin-quantum dot 655 (Strep-Qdot™ 655), or streptavidin-Cy5-plasmonic-fluor, and the nuclei were stained with DAPI. Using an epifluorescence microscope, a signal could not be discerned from the conventional fluorophores, and a very faint signal was observed with Strep-Qdot™ 655 (FIGS. 1C-1D). In contrast, a robust signal was detected with the plasmonic-fluor, and, as expected, there was an increase in TNFα secretion with an increasing LPS concentration (FIG. 1E). Imaging was further performed using a 60× objective. It was observed that the “secretion dot pattern” obtained using the plasmonic fluor correlated well with the morphology of the cell observed in bright field images.

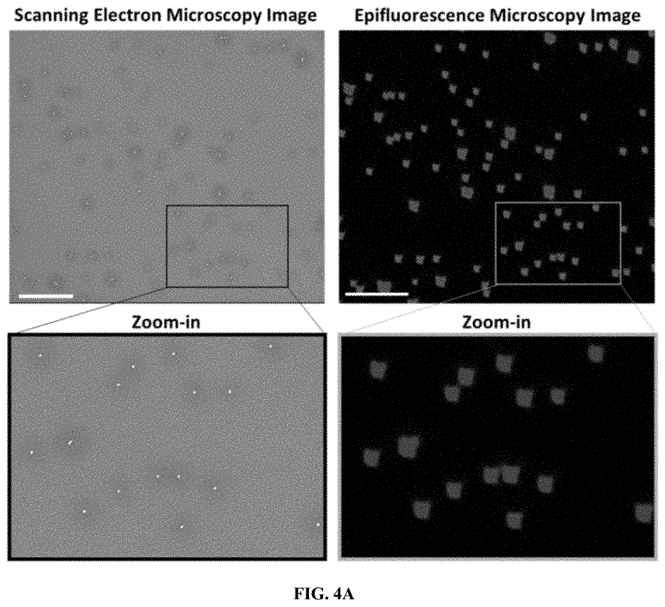

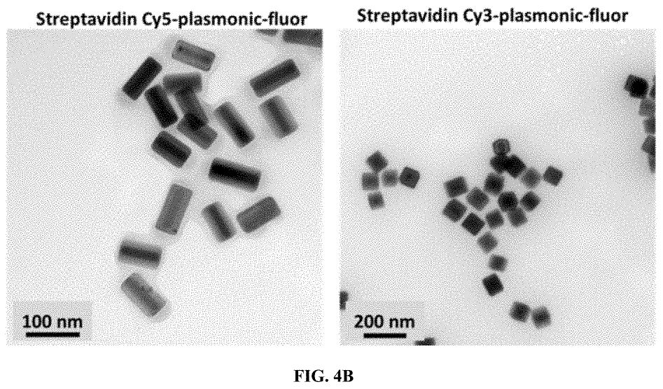

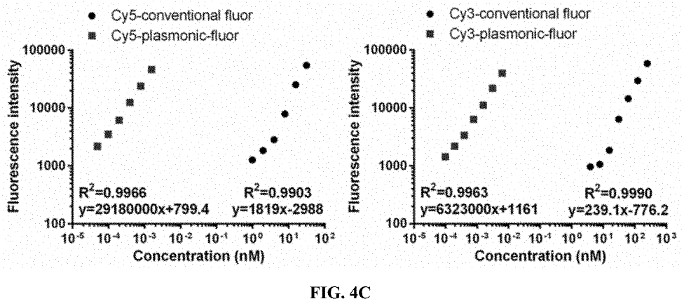

In contrast, no distinct secretion pattern was observed with the other two labels (FIG. 1F). Further, the signal-to-noise ratio of the plasmonic-fluor was nearly 45-fold and 10-fold higher compared to conventional fluorophore and quantum dots, respectively (FIG. 1G). To validate that the fluorescent dots observed are indeed single plasmonic-fluors, the fluorescence image and scanning electron microscopy (SEM) image obtained from the same location were correlated, and a one-to-one correspondence was found (FIG. 4A). Transmission electron microscopy (TEM) images further revealed the size and shape of individual plasmonic-fluors (FIG. 4B). The fluorescence intensity of a defined molar concentration of plasmonic-fluors was further compared with that of conventional fluorophore. The fluorescence intensity increased linearly with increasing concentrations of both conventional fluorophore and plasmonic-fluors (both Cy5 and Cy3) with at least three orders of magnitude difference in molar concentrations for similar fluorescence intensity. Notably, the slope of Cy5-plasmonic-fluor and Cy3-plasmonic-fluor were nearly 16,000- and 26,400-fold steeper than that of Cy5 and Cy3, respectively (FIG. 4C). This large enhancement in the brightness of the plasmonic-fluors compared to their conventional counterparts is critical for the high-resolution images in the Fluoro-dot assay.

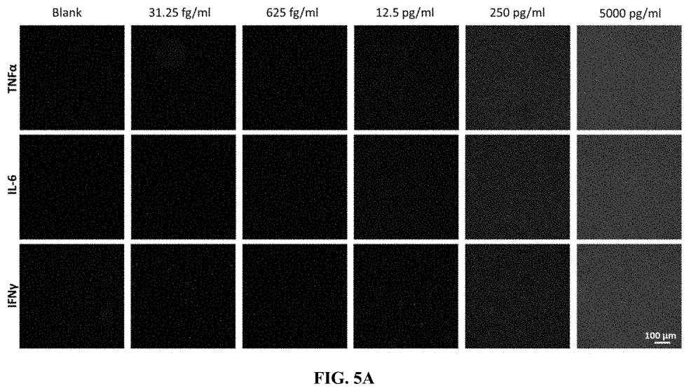

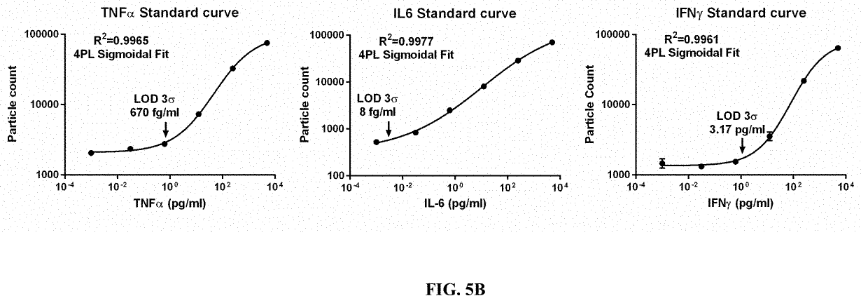

To confirm the analyte dose-dependent increase in the fluorescent dots, a standard fluorescence-based sandwich immunoassay was performed and the particle count correlated with that of concentration of the analyte (TNFα, IL-6, and IFNγ). The particles were counted using a custom-build algorithm. Excellent particle count-dose dependence was observed with a correlation coefficient of >0.99 for all three analytes (limit of detection (LOD) was: TNFα —670 fg/ml; IL-6-8 fg/ml, IFNγ—3.17 μg/ml) (FIGS. 5A-5B). To conclude, using JAWS II DCs, the assay's sensitivity was established by demonstrating its ability to image cytokine secretion.

### Example 3. Fluoro-Dot Assay Detects Protein Secretions at an Ultralow Dose of Stimulant and at Very Early Time Points

One of the fundamental questions in many cell biology studies is the degree of heterogeneity in cellular responses. When cells are subject to a stimulant, it is unknown whether all the cells respond similarly, a small subpopulation of cells exuberantly responds, or a combination of both, and to what extent the heterogeneity depends upon the dose of stimulant. The plasmon-enhanced Fluoro-dot assay was used to examine the dose-response to LPS treatment, with detailed assessment of the kinetics and cell-to-cell heterogeneity of the response. Using JAWS II DCs and LPS as a stimulant, the Fluoro-dot assay revealed no significant secretion of TNFα for an incubation duration of 90 minutes at 2 ng/ml of LPS.

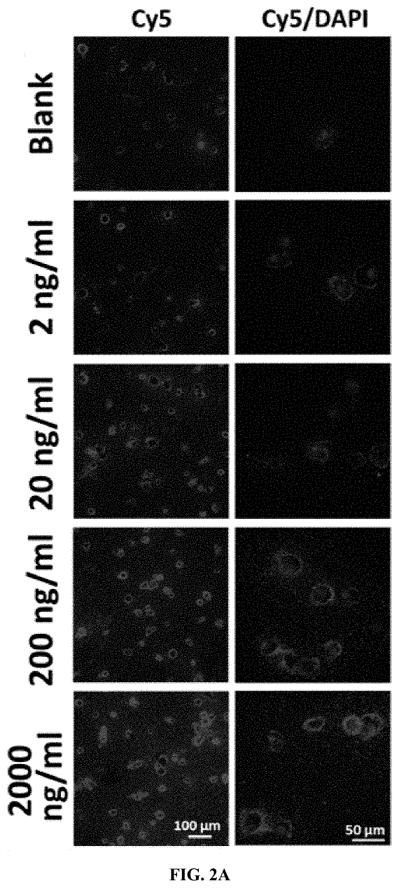

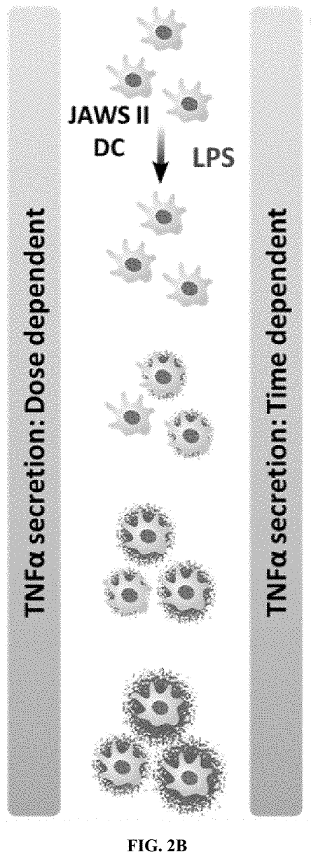

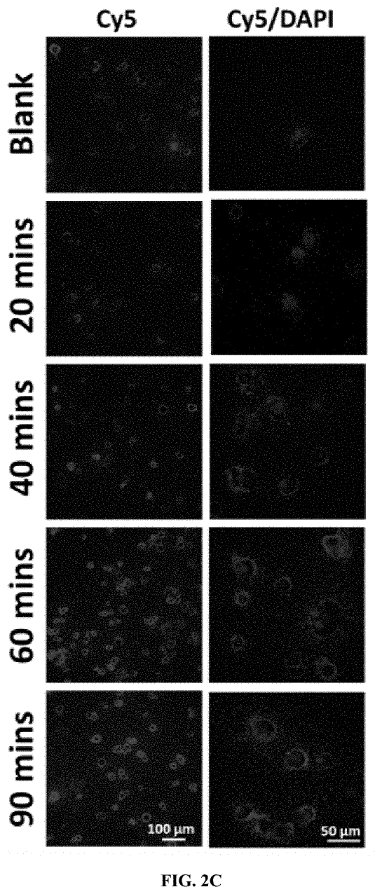

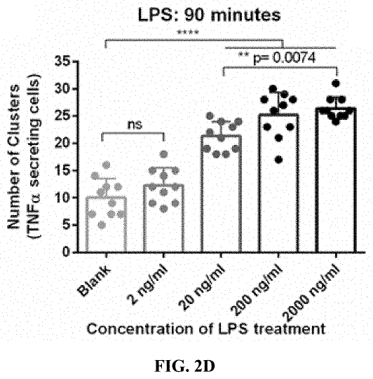

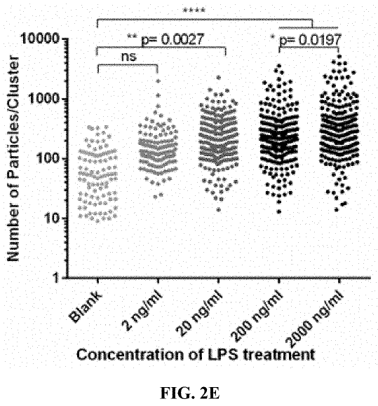

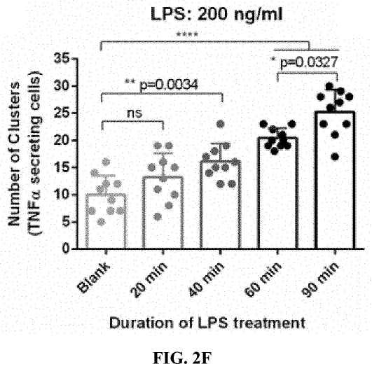

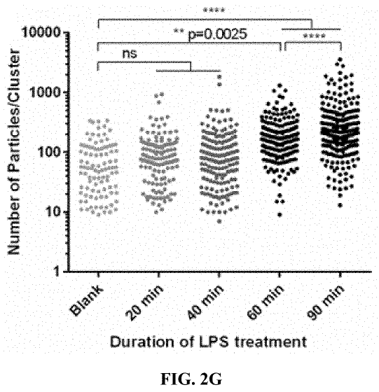

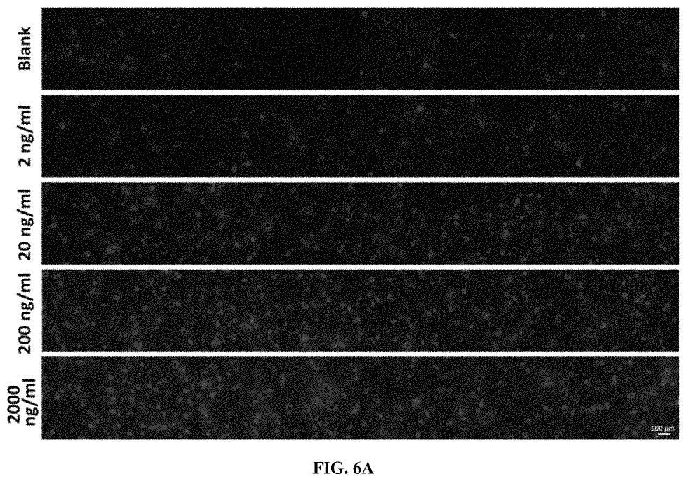

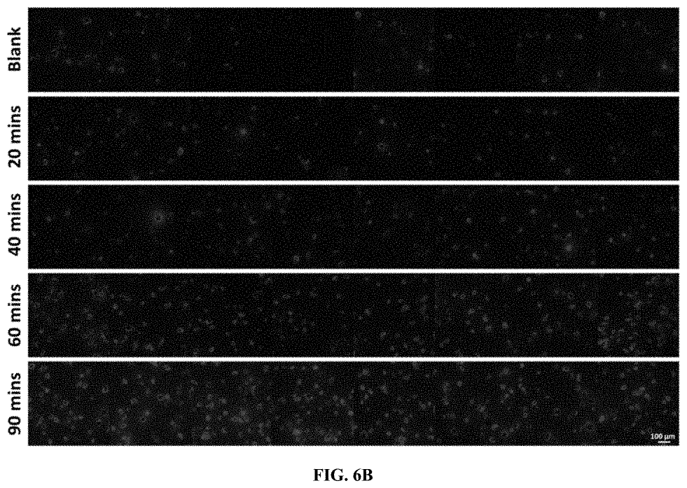

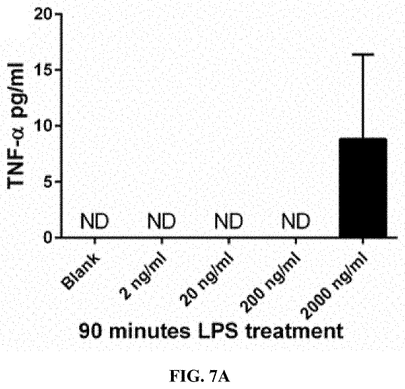

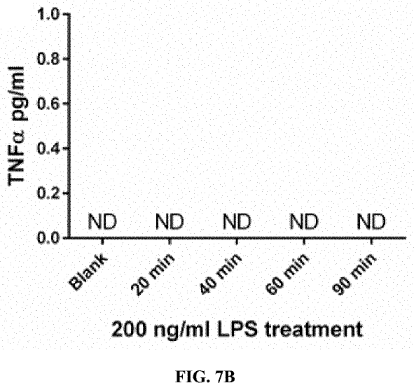

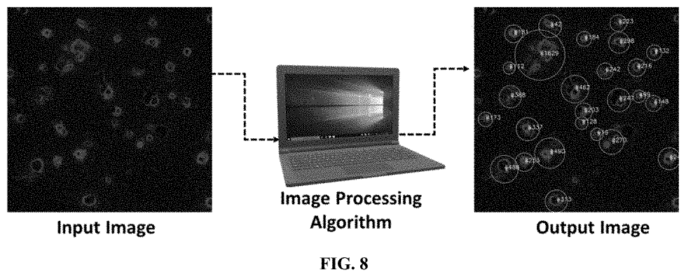

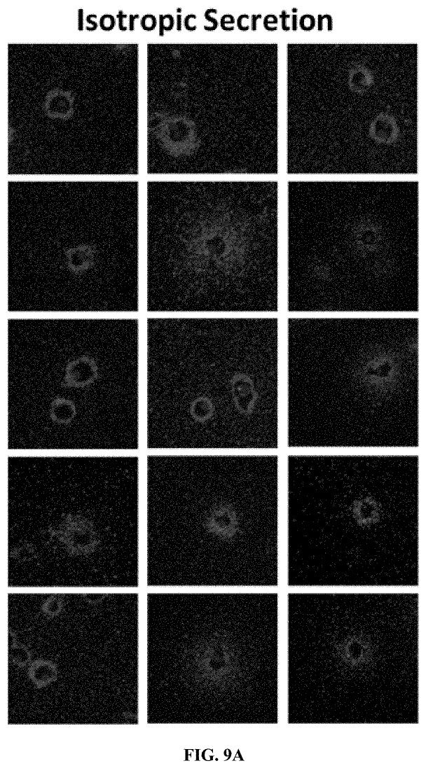

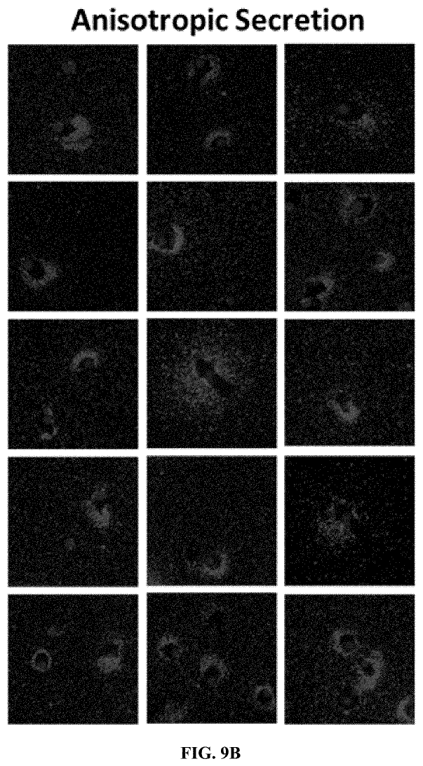

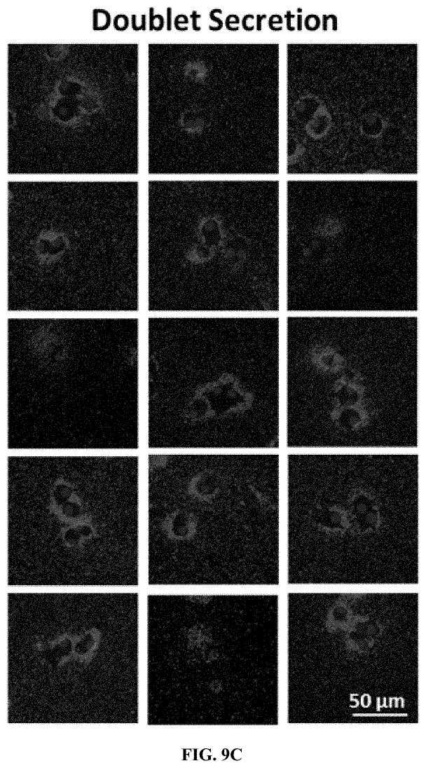

Substantial secretion was observed at 20 ng/ml of LPS, which continued to increase with increasing LPS concentration (FIGS. 2A, 6A). Similarly, using LPS concentration of 200 ng/ml, TNFα secretion was detectable after 40 minutes and increased at longer incubation time (60 and 90 minutes) (FIGS. 2C, 6B). Based on the images, it was observed that both the number of cells secreting TNFα and the amount of TNFα secretion per cell increased with increasing dose and duration of LPS treatment (FIG. 2B). This information cannot be derived from ELISA, which relies on the analysis of the cell culture medium, as the amount of secretion with low dose and short duration of stimulation was below its detection limit (FIGS. 7A-7B). Described herein is an algorithm developed to identify clusters of dots and count both the number of clusters in an image and the number of dots within each cluster (FIG. 8). Using this algorithm, TNFα secreting cells were quantified, as indicated by the number of clusters (FIGS. 2D, 2F), and amount of TNFα secretion from each cell based upon the number of dots (i.e. particles) in each cluster (FIGS. 2E, 2G). The quantitative data corroborated the observations as the difference between treated and untreated cells was only statistically significant when the LPS dose was higher than 20 ng/ml for 90 min incubation or the duration of treatment was more than 40 minutes with 200 ng/ml LPS. Interestingly, it was observed that heterogeneity in cell secretion reflected not only the amount of secretion but also the directionality. While a large fraction of the cells exhibited isotropic secretion all around them, it was observed that a small fraction of the cells secreted TNFα preferentially on one side (FIGS. 9A-9B). “Doublet clusters” from cells that were either seeded close to each other or were undergoing cell division were also observed (FIG. 9C). While the underlying reason behind the anisotropic secretion is a subject of a separate study, the ability of the Fluoro-dot assay to faithfully capture these events further highlights its utility in biomedical research.

### Example 4. Multiplexed Fluoro-Dot Assay for Simultaneous Detection of Multiple Secreted Proteins from a Single Cell

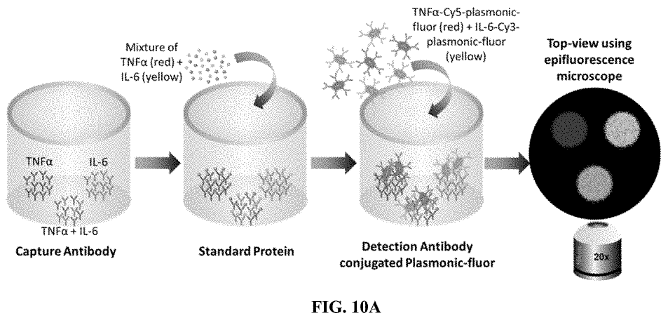

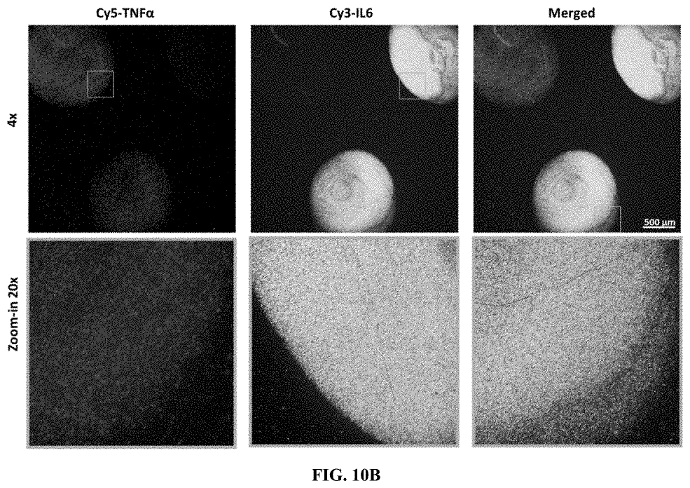

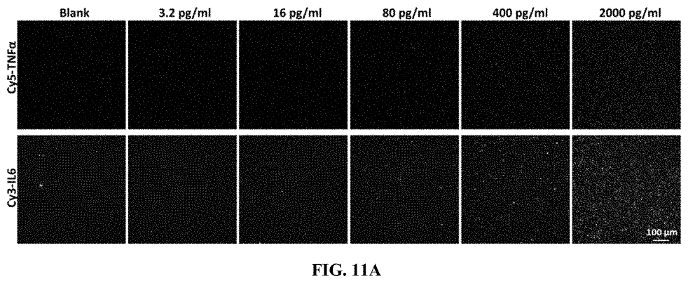

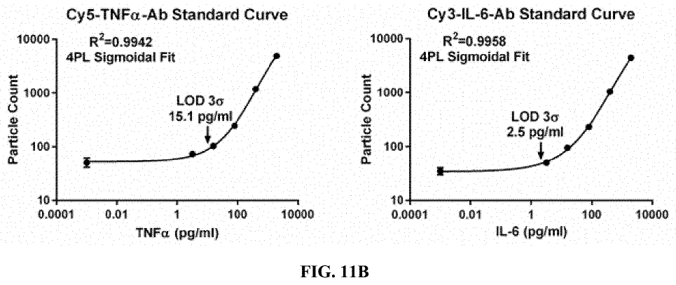

As a proof-of-concept, the feasibility of spectrally multiplexed analysis of two proteins at a single-cell level was investigated. For this purpose, detection antibody was conjugated for TNFα and IL-6 to Cy5-plasmonic-fluors and Cy3-plasmonic-fluors, respectively. The specificity of individual plasmonic-fluors to their respective analyte was confirmed by spotting the capture antibody for analytes (i.e. IL-6 and TNFα) within the same well of a microtiter plate (FIG. 10A). As anticipated, both plasmonic-fluors specifically recognized their respective analytes in single capture antibody and double-capture antibody-coated regions of the plate (FIG. 10B). Further, multiplexed plasmon-enhanced fluorescent immunosorbent assay was performed and a standard curve with a good correlation (R²=0.9942, LOD=15.1 μg/ml for TNFα and R²=0.9958, LOD=2.5 μg/ml for IL-6, respectively) between the particle count and concentration of the protein analyte was observed (FIGS. 11A-11B).

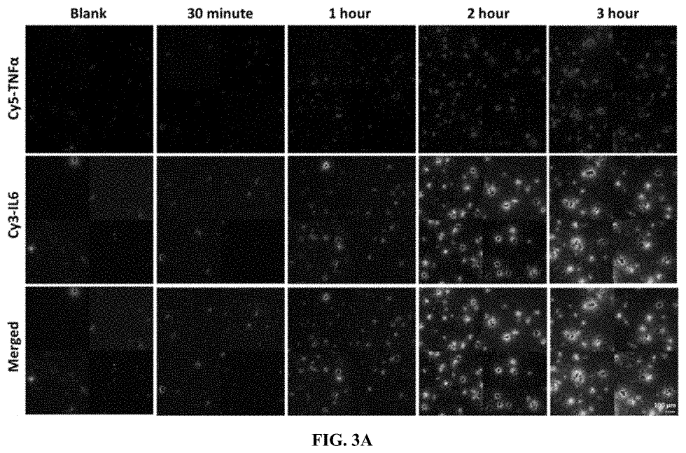

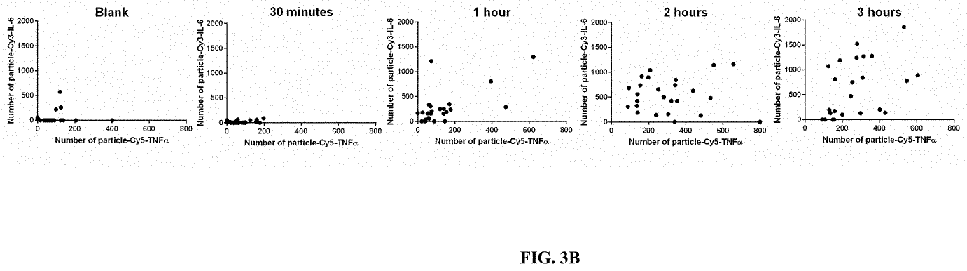

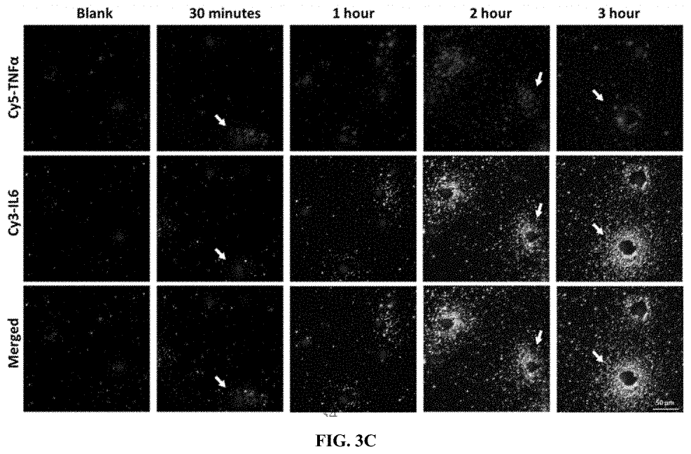

Using LPS-stimulated JAWS II DCs, the concurrent secretion of TNFα and IL-6 at a single-cell level was assessed. An increase in the overall secretion of both cytokines with the increasing duration of LPS incubation from 30 minutes to 3 hours was observed (FIG. 3A). There was significant heterogeneity in the secretion of both cytokines in terms of the amount of secretion (given by the number of particles) and temporal dynamics (FIG. 3B). Interestingly, significant cell-to-cell variation was observed, even after 30 minutes of LPS treatment, such that some cells started secreting TNFα while no secretion for IL-6 was observed for the same cells (FIG. 3C). At longer time durations (2 hours and 3 hours), more predominant IL-6 secretion as compared to TNFα was observed. The observations align with previous findings in which mRNA quantification and ELISA-based assay showed TNFα and IL-6 were produced with distinct kinetics in LPS-treated, activated DCs. Thus, using the multiplexed Fluoro-dot assay, population dynamics of two proteins secreted from single cells were able to be ascertained simultaneously. While as a proof-of-concept cytokine secretion for two proteins has been demonstrated, it is worth noting that the LSPR (localized surface plasmon resonance) wavelength of the plasmonic nanostructures is highly sensitive to its composition, size, and shape (FIG. 4B), and can therefore be tuned over a wide range covering visible to near-infrared wavelength and the plasmonic-fluor can be generated with any molecular fluorophore, thus enabling higher multiplexing capability (up to 6 colors with minimal spectral overlap).

### DISCUSSION

ELISpot is a widely employed bioanalytical method, used in both research and clinical settings for a wide range of applications such as screening antigen-specific immune cells and functional T cells in cancer patients. It is used to assess response to immunotherapy, diagnose tuberculosis (T-SPOT.TB assay), map T cell responses against HIV, detect antibody secreting cells in blood after vaccination, and for functional analysis of circulating tumor cells (CTCs) to understand drug susceptibility. Despite its enormous utility, this method has not undergone significant technological advances in spatial resolution or sensitivity over the past three decades. ELISpot assays thus remain severely limited by the inability to spatially resolve protein secretion, discrepancies in assay read-out, and false negatives in clinical diagnosis.

Herein is introduced a simple, yet powerful, ultra-resolved digital version of ELISpot and FluoroSpot assay called plasmon-enhanced Fluoro-dot assay. The unmet need for an exceptionally bright fluorescent tag capable of detecting single-molecule fluorescence is served by plasmonic-fluors, which outperform conventional fluorophores and quantum dots. Using plasmonic-fluors, the limit-of-detection of the Fluoro-dot assay is improved by more than two orders of magnitude compared to conventional biolabels. The signal is digitized and the fluorescent dots can be counted, in contrast to integrating the signal as in the case of ELISpot and FluoroSpot, which can be considered as analog version of this assay. This method will provide deeper insights into how the mechanotransduction, polarization, and adhesion properties of cells correlates with single-cell protein secretion, and application of this method for studying inflammasome-mediated protein secretion in diverse cell types including immune cells and endothelial cells.

Since heterogeneity is a fundamental characteristic of cellular systems, single-cell technologies and measurements are of immense value for an in-depth understanding of individual cells, facilitating disease model development, drug discovery, and meaningful biological insights beyond ensemble population averages. The Fluoro-dot assay enables the visualization of these parameters (heterogeneity, directionality) and can be implemented for better understanding of host-pathogen interaction, neuronal secretions, and tumor cytotoxicity. When compared to any of the conventionally used approaches, Fluoro-dot assay can lead to transformative advances in single-cell secretion studies as it is versatile, low-cost, and is readily adaptable in any laboratory setting with regularly used supplies and reagents (glass-bottom 96-well plate, ELISA reagents), commercially available plasmonic-fluor (Auragent Bioscience LLC), and a standard epifluorescence microscope. Considering the evolving landscape of cellular traits, this method holds the potential to provide a comprehensive understanding of single-cell secretome.

### CONCLUSIONS

Existing bionalaytical methods are limited to the analysis of cell culture supernatant for understanding the changes in the protein secretions upon external stimulus. Naturally, the concentrations of the target proteins in cell culture supernatants represent an ensemble average of the entire cell population (in vitro), thus masking the cell-to-cell heterogeneity and, more importantly, the locoregional correlation between biophysical stimulation and biochemical response. The present disclosure introduces a novel bioanalytical method that reveals the protein secretion patterns at single cell level to understand the biophysical and biochemical correlation.

JAWSII dendritic cells (JAWSII DC) were seeded on substrates coated with capture antibody for TNFα. The cells were treated with different concentrations (20, 200, 2000 ng/ml) of lipopolysaccharide (LPS) and after 90 minutes the cells were washed with phosphate buffered saline containing 0.05% Tween-20 (PBST). The cells were treated with biotinylated detection antibody, followed by streptavidin conjugated conventional fluor Cy5, quantum dots 655 and Cy5-plasmonic-fluor. As seen in FIGS. 1A-1G, the plasmonic-fluor enabled detection of secreted TNFα after treatment with LPS at an individual cell level. Plasmonic-fluors clearly revealed the cell-to-cell heterogeneity in terms of TNFα secretion, while there was no discernable signal in wells treated with conventional fluorophore or quantum dots. An image processing algorithm was also developed, which enabled quantification of the cell secretion at individual cell level and detection of 2 cytokines simultaneously using multi-colored plasmonic-fluors.

Accordingly, demonstrated herein is a platform technology that enables observing correlation between chemical stimulus (e.g., immunostimulating agent) and biological response (e.g., protein secretion) of cultured cells in vitro. In contrast to existing approaches that rely on conventional fluorophores or enzymatic reactions, described herein is an ultrabright nanolabel based on plasmon-enhanced fluorescence for ultrasensitive, multiplexed detection and quantification of secreted proteins. Colorimetric sensing based on enzymatic reactions is not sufficiently sensitive for the detection of low abundance biomarkers and, more importantly, is unsuitable for locoregional mapping of the secreted proteins. The fluorescence signal from conventional fluorophores is too weak, making them unsuitable for detection of secreted proteins even at relatively high concentrations (>200 pg/ml). The use of plasmonic-fluor as an ultrabright nanolabel overcomes these challenges and enables the detection of low abundant secreted proteins at an early time-point.

This written description uses examples to illustrate the present disclosure, including the best mode, and also to enable any person skilled in the art to practice the disclosure, including making and using any compositions or systems and performing any incorporated methods. The patentable scope of the disclosure is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed disclosure. The term “consisting essentially of” occupies a middle ground between “comprising” and “consisting of”.

Where a disclosure or a portion thereof is defined with an open-ended term such as “comprising,” it should be readily understood that (unless otherwise stated) the description should be interpreted to also describe such a disclosure using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the disclosure are intended to be nonrestrictive regarding the number of instances (i.e., occurrences) of the element or component. Therefore “a” or “an” should be read to include one or at least one, and the singular word form of the element or component also includes the plural unless the number is obviously meant to be singular.

What is claimed is:

1. A method for detecting cellular protein secretion, the method comprising:
adding at least one stimulant to a sample comprising at least one cell fixed on a substrate, wherein the at least one stimulant stimulates the at least one cell to secrete at least one protein;
capturing the at least one protein secreted by the at least one cell;
adding at least one detection antibody to the sample;
adding at least one plasmonic-fluor to the sample; and
measuring at least one fluorescence signal from the sample.

2. The method of claim 1, wherein the at least one stimulant is selected from the group consisting of toll-like receptor 4 (TLR4) agonists, lipopolysaccharide (LPS), and combinations thereof.

3. The method of claim 1, wherein the at least one detection antibody is biotinylated.

4. The method of claim 1, wherein the at least one plasmonic-fluor is streptavidin-conjugated.

5. The method of claim 1, wherein the at least one plasmonic-fluor comprises a fluorescent label selected from the group consisting of Cy3, Cy5, and combinations thereof.

6. The method of claim 1, wherein the substrate is selected from the group consisting of glass substrates, plastic substrates, well-plates, and combinations thereof.

7. The method of claim 1, wherein the method step of measuring at least one fluorescence signal from the sample comprises measuring the at least one fluorescence signal with a technique selected from the group consisting of fluorescence microscopy, epifluorescence microscopy, and combinations thereof.

8. The method of claim 1, wherein the method step of measuring at least one fluorescence signal from the sample comprises measuring a plurality of fluorescence signals from a plurality of fluorescent dots.

9. The method of claim 1, wherein the at least one cell is selected from the group consisting of dendritic cells, JAWS II cells, and combinations thereof.

10. The method of claim 1, wherein the at least one protein secreted by the at least one cell is selected from the group consisting of cytokines, TNFα, IL-1β, IFN-α/β, IL-6, IFNγ, and combinations thereof.

11. The method of claim 1, wherein the method further comprises measuring at least one spatial signal from the sample, wherein the at least one spatial signal is measured with a technique selected from the group consisting of microscopy, scanning electron microscopy (SEM), transmission electron microscopy (TEM), and combinations thereof.

12. The method of claim 1, wherein the method further comprises incubating the sample.

13. A kit comprising:
a sample comprising a substrate, wherein the substrate optionally comprises at least one cell affixed thereon;
at least one stimulant;
at least one detection antibody;
at least one plasmonic-fluor; and
written instructions for detecting cellular protein secretion, comprising the steps of:
optionally affixing at least one cell on the substrate;
adding the at least one stimulant to the sample, wherein the sample comprises at least one cell fixed on the substrate, wherein the at least one stimulant stimulates the at least one cell to secrete at least one protein;
capturing the at least one protein secreted by the at least one cell;
adding the at least one detection antibody to the sample;
adding the at least one plasmonic-fluor to the sample; and
measuring at least one fluorescence signal from the sample.

14. The kit of claim 13, wherein the at least one stimulant is selected from the group consisting of toll-like receptor 4 (TLR4) agonists, lipopolysaccharide (LPS), and combinations thereof.

15. The kit of claim 13, wherein the at least one detection antibody is biotinylated.

16. The kit of claim 13, wherein the at least one plasmonic-fluor is streptavidin-conjugated.

17. The kit of claim 13, wherein the at least one plasmonic-fluor comprises a fluorescent label selected from the group consisting of Cy3, Cy5, and combinations thereof.

18. The kit of claim 13, wherein the substrate is selected from the group consisting of glass substrates, plastic substrates, well-plates, and combinations thereof.

19. The kit of claim 13, wherein the at least one cell is selected from the group consisting of dendritic cells, JAWS II cells, and combinations thereof.

20. The kit of claim 13, wherein the at least one protein secreted by the at least one cell is selected from the group consisting of cytokines, TNFα, IL-1β, IFN-α/β, IL-6, IFNγ, and combinations thereof.
